(19) United States
(12) Patent Application Publication
(10) Pub. No.: US 2022/0211654 A1
(43) Pub. Date: Jul. 7, 2022

(54) MATERIALS AND METHODS FOR TREATING AGE-RELATED MACULAR DEGENERATION

(71) Applicant: THE REGENTS OF THE UNIVERSITY OF CALIFORNIA, Oakland, CA (US)

(72) Inventors: Howard J. Federoff, lrvine, CA (US); Sudhakar Raja Subramaniam, lrvine, CA (US); Sonali Nashine, lrvine, CA (US); M. Cristina Kenney, Malibu, CA (US)

(21) Appl. No.: 17/609,054

(22) PCT Filed: May 6, 2020
(86) PCT No.: PCT/US20/31610

§ 371 Date: Nov. 5, 2021

Related U.S. Application Data
(60) Provisional application No. 62/843,624, filed on May 6, 2019.

(51) Int. Cl. A61K 31/216; A61K 31/352; A61K 31/4184; A61P 27/02

(52) U.S. Cl. CPC A61K 31/216; A61P 27/02; A61K 31/4184; A61K 31/352

(57) ABSTRACT

Described herein are materials and methods for the treatment of age-related macular degeneration by administering a combination of fenofibrate and an esterase inhibitor (e.g., kaempferol or telmisartan).

### CROSS REFERENCE TO RELATED APPLICATIONS

The present application claims the priority benefit of U.S. Provisional Patent Application No. 62/843,624, filed May 6, 2019, hereby incorporated by reference in its entirety.

### FIELD OF THE INVENTION

The present disclosure is directed to methods of treating age-related macular degeneration in a subject in need thereof.

### BACKGROUND

Mitochondrial (mt) DNA damage arising due to mutations or oxidative stress has long been implicated in the development of AMD (4). Mitochondrial DNA damage induces ARPE-19 cells to secrete pro-inflammatory cytokines associated with onset and progression of AMD (5). Macular RPE cells from aged and AMD human donor eyes have higher frequencies of mtDNA lesions and mtDNA genomic heteroplasmic mutations, compared to their age-matched controls. AMD severity has been associated with declined expression of a DNA repair enzyme OGG1, which is involved in excision repair of oxidatively-damaged DNA. Accumulation of mtDNA lesions and reduced DNA repair capacity contribute to loss of RPE cells in AMD and aging retina (6). Therefore, several mitochondria-targeting therapeutic molecules have been identified in the hope of rescuing mtDNA and subsequently RPE cells in AMD. For instance, a mitochondria-targeted antioxidant SkQ1 prevents AMD progression in an in vivo model of AMD (7). A mitochondria-targeting peptide called MTP-131 (Bendavia) targets cardiolipin and improves mitochondrial function (8). Furthermore, Humanin G—a more potent variant of a mitochondrial-derived peptide Humanin, rescues AMD RPE cells in vitro (9).

Age-related macular degeneration (AMD) is a sudden worsening and distortion of central vision that progresses rapidly, typically with a course of only weeks or months. AMD is characterized by abnormalities in the macular area. The central area (or fovea) of the macula contains the highest density of cone photoreceptors in the retina and mediates high-acuity vision. The disease typically has a preclinical, asymptomatic phase, in which extracellular waste material accumulates in the space between the basement membrane (Bruch's membrane) and the epithelial layer, forming yellow-white spots known as drusen. Advanced forms of AMD includes both dry and wet (or “neovascular”) AMD. The dry form of AMD is more prevalent, but the wet form occurs simultaneously with the dry form in about 15% of cases. Dry AMD is characterized by progressive apoptosis of cells in the epithelial layer, in the overlying photoreceptor cells and in the underlying cells in the choroidal capillary layer. Wet AMD is characterized by choroidal neovascularization with vascular leakage into subretinal spaces.

AMD impairs central vision that is required for reading, driving, face recognition and fine visual tasks. Neurosensory detachment, retinal hemorrhages and retinal scarring gradually result in decreased visual function of photoreceptors in the central vision, eventually resulting in legal blindness, with preservation of peripheral vision. AMD is the most common cause of blindness among the elderly. Subjects with a family history of AMD and those who smoke have a higher risk than non-smokers and those with no family history. Nevertheless, subjects who have favorable risk profiles also develop the disease. Current therapeutic efforts and clinical trials are primarily aimed at halting the growth of the neovascular membrane in wet AMD, e.g., using angiogenesis (VEGF-A) inhibitors, laser photocoagulation and/or photodynamic therapy. Antioxidants can retard the progression of the disease.

### SUMMARY

In one aspect, described herein is a method for treating age-related macular degeneration (AMD) in a subject in need thereof comprising administering fenofibrate to the subject. In some embodiments, the method further comprises administering an esterase inhibitor to the subject. In some embodiments, the esterase inhibitor is kaempferol or telmisartan. In some embodiments, the esterase inhibitor is kampferol. In some embodiments, the esterase inhibitor is kaempferol. The fenofibrate and esterase inhibitor (e.g., kaempferol or telmisartan) can be administered concomitantly or sequentially.

In some embodiments, the method comprises determining if the subject receiving treatment has a reduced level of PGC-1a expression as compared to a control subject.

In another aspect, described herein is a method of decreasing inflammation in a retinal pigment epithelium (RPE) cybrid cell in a subject in need thereof, comprising administering fenofribrate to the subject. In some embodiments, the method further comprises administering an esterase inhibitor (e.g., kaempferol or telmisartan) to the subject. In some embodiments, the subject is suffering from age-related macular degeneration (AMD). In various embodiments, the age-related macular degeneration is wet or dry age-related macular degeneration. In some embodiments, the subject does not have diabetes.

In another aspect, described herein is a method of inducing PGC-1α expression in a retinal pigment epithelium (RPE) cybrid cell comprising contacting the cell with fenofibrate. In some embodiments, the method further comprises administering an esterase inhibitor (e.g., kaempferol or telmisartan) to the subject.

In another aspect, described herein is a method of increasing mitochondrial load in a retinal pigment epithelium (RPE) cybrid cell comprising contacting the cell with fenofibrate. In some embodiments, the method further comprises administering an esterase inhibitor (e.g., kaempferol or telmisartan) to the subject.

### BRIEF DESCRIPTION OF THE FIGURES

FIGS. 1A-1E shows that fenofibrate regulates the mitochondrial biogenesis pathway. Quantitative RT-PCR (SYBR green) to measure the expression of markers of the mitochondrial biogenesis pathway such as PGC-1α (FIG. 1A), NRF-1 (FIG. 1B), NRF-2 (FIG. 1C), PPAR-α (FIG. 1D), and PPAR-γ (FIG. 1E). fenofibrate (PU-91)-treated AMD cybrids had higher gene expression levels of all the above-mentioned markers (p<0.05, n=4-5). Data are represented as mean±SEM, normalized to untreated (UN) AMD cybrids, which were assigned a value of 1. Student's t-test was used to measure statistical differences.

FIGS. 2A-2E show that fenofibrate regulates mitochondrial function. The fluorometric JC-1 assay and MitoSOX assay were used to measure mitochondrial membrane potential and mitochondrial superoxide production respectively. Treatment with fenofibrate (PU-91) led to elevated mitochondrial membrane potential (FIG. 2A) (p<0.05, n=3) and reduced mitochondrial superoxide production (FIG. 2B) (p<0.05, n=3) in AMD cybrids compared to the untreated group. Furthermore, fenofibrate (PU-91)-treated AMD cybrids showed up-regulation of the mitochondrial superoxide dismutase, SOD2 gene (FIG. 2C) (p<0.05, n=5) and reduced expression of HIF1α gene (FIG. 2D) (p<0.05, n=3-4). Data are represented as mean±SEM, normalized to untreated (UN) AMD cybrids, which were assigned a value of 1. Student's t-test was used to measure statistical differences. Fenofibrate up-regulated MT-RNR2 gene. Using TaqMan probe for the MT-RNR2 gene (FIG. 2E), qRT-PCR analysis revealed that fenofibrate increases MT-RNR2 gene expression by 104% compared to untreated control (p<0.05, n=5). Data are represented as mean±SEM, normalized to untreated (UN) AMD cybrids, which were assigned a value of 1. Student's t-test was used to measure statistical differences.

FIGS. 3A-3C show that fenofibrate (PU-91) regulates apoptotic cell death. Using the MTT assay, it was observed that fenofibrate (PU-91)-treated AMD cybrids had a higher number of viable cells compared to the untreated group (FIG. 3A) (p<0.05, n=4). qRT-PCR analysis showed down-regulation of apoptotic genes such as Caspase-3 (FIG. 3AB) (p<0.05, n=4) and BAX (FIG. 3B) (p<0.05, n=4) in AMD cybrids treated with fenofibrate (PU-91). Data are represented as mean±SEM, normalized to untreated (UN) AMD cybrids, which were assigned a value of 1. Student's t-test was used to measure statistical differences.

FIGS. 4A-4B show that fenofibrate alters mitochondrial GFP fluorescence intensity. Untreated and fenofibrte-treated cybrids were stained with CellLight mitochondrial GFP stain followed by confocal imaging of cells. FIG. 4A shows representative bright-field, DAPI, mtGFP, and overlay (DAPI+mtGFP) confocal images. Fenofibrate (PU-91)-treated AMD cybrids had a drastic increase in mtGFP fluorescence intensity compared to the untreated group (FIG. 4B) (p<0.05, n=3). Data are represented as mean±SEM, normalized to untreated (UN) AMD cybrids, which were assigned a value of 1. Student's t-test was used to measure statistical differences.

FIGS. 5A-5C show that fenofibrate (PU-91) regulates inflammation and complement. qRT-PCR analysis showed lower gene expression of inflammation markers such as IFNB1 (FIG. 5A) (p<0.05, n=4), IL-18 (FIG. 5B) (p<0.05, n=4) in fenofibrate (PU-91)-treated AMD cybrids. However, fenofibrate up-regulated the complement inhibitor CFH gene (FIG. 5C) (p<0.05, n=3-4). Data are represented as mean±SEM, normalized to untreated (UN) AMD cybrids, which were assigned a value of 1. Student's t-test was used to measure statistical differences.

FIGS. 6A-6D show the effect of fenofibrate (PU-91)+kaempferol (E-12)/telmisartan (E-78) on cell viability. This figure shows cell viability differences using MTT assay in AMD cells treated with fenofibrate (PU-91)+kaempferol (E-12) (FIGS. 6A and 6B)/telmisartan (E-78 (FIGS. 6C and 6D) at 48 hr and 72 hr time points. Data (n=3) are represented as mean±SEM, normalized to untreated (UN) AMD cybrids, which were assigned a value of 1. One-way ANOVA and Student's t-test were used to measure statistical differences (p<0.05).

FIGS. 7A-7E show the effect of fenofibrate (PU-91)+kaempferol (E-12) on gene expression. qRT-PCR analysis showed differential expression of PGC-1α (FIG. 7A), Caspase-3 (FIG. 7B), IL-18 (FIG. 7C), VEGF (FIG. 7D), SOD2 (FIG. 7E) genes in AMD RPE cells at the 72 hr time point. Data (n=3) are represented as mean±SEM, normalized to untreated (UN) AMD cybrids, which were assigned a value of 1. One-way ANOVA and Student's t-test was used to measure statistical differences (p<0.05).

FIGS. 8A-8E show the effect of fenofibrate (PU-91)+telmisartan (E-78) on gene expression. qRT-PCR analysis showed differential expression of PGC-1α (FIG. 8A), Caspase-3 (FIG. 8B), IL-18 (FIG. 8C), VEGF (FIG. 8D), SOD2 (FIG. 8E) genes in AMD RPE cells at the 72 hr time point. Data (n=3) are represented as mean±SEM, normalized to untreated (UN) AMD cybrids, which were assigned a value of 1. One-way ANOVA and Student's t-test was used to measure statistical differences (p<0.05).

### DETAILED DESCRIPTION

The present disclosure provides a method for treating age-related macular degeneration (AMD) in a subject in need thereof comprising administering fenofibrate, optionally in combination with an esterase inhibitor such as kaempferol or telmisartan, to the subject.

As demonstrated in the Examples, gene expression analyses revealed significant upregulation of mitochondrial biogenesis pathway genes—PGC-1a, NRF-1, NRF-2, PPAR-α, and PPAR-γ in fenofibrate-treated AMD cybrids. Since all cybrids have identical nuclei and differ only in mitochondrial DNA (derived from AMD patients), these results suggest that fenofibrate can influence mtDNA-mediated up-regulation of nucleus-encoded markers of mitochondrial biogenesis.

Unless otherwise defined, all technical and scientific terms used herein have the same meaning as commonly understood by one of ordinary skill in the art to which this invention belongs.

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

As used herein, the following terms have the meanings ascribed to them unless specified otherwise.

### Definitions

As used herein, “age-related macular degeneration” or “AMD” includes both early, intermediate, and advanced AMD. Patients with early AMD are usually asymptomatic, and present clinically with yellowish drusen seen underneath the retinal pigment epithelium, with areas of mottled retinal pigment epithelium hyperpigmentation and hypopigmentation. AMD includes both dry and wet AMD.

As used herein, a “subject” is a mammal, e.g., human, canine, feline, ovine, primate, equine, porcine, caprine, camelid, avian, bovine, and murine organisms. Typically, the subject is a human.

The term “control” is meant a value from a subject lacking the age-related macular degenerative disease or a known control value exemplary of a population of subjects lacking the maculular degenerative disease, or with baseline or healthy subject levels of a biomarker such as PGC1α protein. In some cases as described above, a control value can be from the same subject before the onset of a neurodegenerative disease or before the beginning of therapy therefor.

The terms “treat”, “treating”, and “treatment” refer to a method of reducing or delaying one or more effects or symptoms of age-related macular degeneration (AMD). The subject can be diagnosed with the disease. Treatment can also refer to a method of reducing the underlying pathology rather than just the symptoms. The effect of the administration to the subject can have the effect of but is not limited to reducing one or more symptoms of the macular degenerative disease or disorder, a reduction in the severity of the disease or injury, the complete ablation of the AMD, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

The term “prevent”, “preventing”, or “prevention” is meant a method of precluding, delaying, averting, obviating, forestalling, stopping, or hindering the onset, incidence, severity, or recurrence of the AMD or one or more symptoms thereof. For example, the disclosed method is considered to be a prevention if there is a reduction or delay in onset, incidence, severity, or recurrence of AMD or one or more symptoms of AMD (e.g., blurred vision, shadowy areas in central vision, sensitivity to glare, difficulty reading in low light levels, difficulty watching television, difficulty using a computer, decreased sensitivity to color tests, finding distortion of straight lines so they appear wavy) in a subject susceptible to AMD as compared to control subjects susceptible to AMD that did not receive fenofibrate, optionally in combination with the esterase inhibitor (e.g., kaempferol or telmisartan). The disclosed method is also considered to be a prevention if there is a reduction or delay in onset, incidence, severity, or recurrence of AMD or one or more symptoms of AMD in a subject susceptible to AMD after receiving fenofibrate or analog thereof with an esterase inhibitor (e.g., kaempferol or telmisartan) as compared to the subject's progression prior to receiving treatment. Thus, the reduction or delay in onset, incidence, severity, or recurrence of age-related macular degeneration can be about a 10, 20, 30, 40, 50, 60, 70, 80, 90, 100%, or any amount of reduction in between.

The term “subject” as used herein means an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

The present disclosure is based on the discovery fenofibrate protects age-related macular degeneration (AMD) ARPE-19 transmitochondrial cybrid cells by preserving mitochondrial health, reducing apoptotic cell loss, and inducing transcription of the MDP-coding MT-RNR2 gene.

In one aspect, described herein is a method of treating age-related macular degeneration in a subject comprising administering fenofibrate or analog thereof to a subject in need thereof. In some embodiments, the method further comprises administering an esterase inhibitor to the subject. Exemplary esterase inhibitor include, but are not limited to kaempferol and telmisartan.

In some embodiments, the esterase inhibitor is kaempferol. In some embodiments, the esterase inhibitor is telmisartan. In some embodiments, the fenofibrate or analog thereof and kaempferol are preferably administered at a fixed molar ratio. In some embodiments, the molar ratio of fenofibrate or analog thereof to kaempferol is 1.5:1, 2:1, 3:1, or 4:1.

Age-related macular degeneration is a progressive disease that can lead to permanent loss of vision. AMD is distinguished from acute retinal damage in that the disease is age-related and generally starts with accumulation of drusen underneath the retinal pigment epithelium (RPE), progressively causing RPE dysfunction and ultimately leading to photoreceptor loss. AMD can, but not always, advance very slowly and vision loss may not occur for a long time. In others, AMD can progress faster and may lead to a loss of vision in one or both eyes. AMD differs from acute retinal damage induced either by injuries or sun damage, neither of which are caused by aging or marked by the presence of drusen.

In some embodiments, the AMD treated according to the methods described herein is dry AMD. In other embodiments, the AMD treated according to the methods described herein is wet AMD. The term “dry AMD” is well known in the art and is used to mean the condition of age-related macular degeneration marked by the presence of drusen, alterations in retinal pigment epithelium (RPE), accumulation of immune cells such as macrophages and microglia, thickening of Bruch's membrane (including excessive cholesterol and calcium accumulation therein), general atrophy, alterations in the choriocapillaris, degeneration of photoreceptors, and cell death. The appearance of drusen is generally considered one of the first detectable symptoms of AMD, in particular dry AMD. One of skill in the art would understand and be able to readily identify drusen in a subject or a tissue sample taken from a subject. Drusen are deposits that typically comprise acute phase proteins, such as but not limited to, C-reactive protein, vitronectin, a-antichymotrypsin, amyloid P component, and fibrinogen, as well as complement pathway components, such as but not limited to C3, C5 and C5b-9 complex as well as apolipoproteins B and E, mucopolysaccarides, lipids, mannose, crystallins, immunoglobulins, and sialic acid.

Wet AMD, on the other hand, is also well known and is marked by abnormal choroidal blood vessel growth in region of the macula. Ultimately, bleeding and protein leakage can occur through these newly formed blood vessels, which causes loss or photoreceptors and subsequent vision damage. Wet AMD almost always begins with dry AMD, although not all instances of dry AMD will progress to wet AMD.

Patients usually develop rapid visual loss when neovascular AMD occurs. Typically, patients describe sudden worsening of central vision with distortion of straight lines (metamorphopsia) or a dark patch in their central vision (scotoma), or both. In geographic atrophy, there is slower progressive loss of vision over many years. Clinically, there is a sharply demarcated area of depigmentation showing retinal pigment epithelium atrophy. Neovascular AMD is characterised by subretinal or intraretinal fluid and haemorrhage; occasionally, the choroidal neovascularisation complex can be seen clinically. “Advanced AMD” includes both dry AMD and wet AMD (wet AMD is also referred to as neovascular AMD). Subjects with advanced AMD are those who can be categorized in Category 4 according to the AREDS classification.

Various AMD classification schemes have been developed. The Age-Related Eye Disease Study (AREDS) classified age-related macular degeneration into four categories as follows (AREDS Report No 8, Arch Ophthalmol, 2001, 119, 1417-36):
Category 1: None or a few small drusen (<63 μm in diameter).
Category 2: Any or all of the following: multiple small drusen, few intermediate drusen (63-124 μm in diameter), or retinal pigment epithelium abnormalities.
Category 3: Any or all of the following: extensive intermediate drusen, and at least one large drusen (>125 μm in diameter, roughly equivalent to the size of the retinal vein at the rim of the optic disc), and geographic atrophy not involving the fovea.
Category 4: Geographic atrophy involving the fovea or any of the features of neovascular age-related macular degeneration, and visual loss presumed to be due to age-related macular degeneration. Although not part of this classification, advanced AMD might also include the involutional, atrophic stage of neovascular AMD that is not amenable to further treatment.

In the AREDS, the 5-year risk of developing advanced AMD in at least one eye in control participants was 1.3% in eyes in Category 2, 18.3% in those in Category 3, and 43.9% in those in Category 4.

Polypoidal choroidal vasculopathy is difficult to distinguish clinically from choroidal neovascularisation. Occasionally, orange, bulging dilatations might be visible under the retina. However, polypoidal choroidal vasculopathy more commonly presents with recurrent serous and haemorrhagic retinal pigment epithelium detachments. Retinal angiomatous proliferation is characterised clinically by signs of haemorrhage, oedema, and exudates within the retinal layers in addition to other typical signs of choroidal neovascularisation. In some cases, the anastomosis between the retinal and subretinal new vessels might be visible.

Fenofibrate

Fenofibrate is a fibrate compound, previously used in the treatment of endogenous hyperlipidemias, hypercholesterolemias and hypertriglyceridemias. The preparation of fenofibrate is disclosed in U.S. Pat. No. 4,058,552, the disclosure of which is incorporated herein by reference in its entirety. Fenofibric acid is the active metabolite of fenofibrate. Fenofibrate is not soluble in water, which limits its absorption in the gastrointestinal (GI) tract. Alternative formulations and strategies have been used to overcome this problem. See U.S. Pat. Nos. 4,800,079 and 4,895,726 (micronized fenofibrate); U.S. Pat. No. 6,277,405 (micronized fenofibrate in a tablet or in the form of granules inside a capsule); U.S. Pat. No. 6,074,670 (the immediate release of micronized fenofibrate in a solid state; U.S. Pat. No. 5,880,148 (combination of fenofibrate and vitamin E); U.S. Pat. No. 5,827,536 (diethylene glycol monoethyl ether (DGME) as solubilizer for fenofibrate); and U.S. Pat. No. 5,545,628 (the combination of fenofibrate with one or more polyglycolyzed glycerides), all of which are incorporated herein in their entireties by this reference. Numerous other derivatives, analogs and formulations are known to one of skill in the art. For example, other esters of p-carbonylphenoxy-isobutyric acids as described in U.S. Pat. No. 4,058,552, which is incorporated herein by reference in its entirety, can be used. Fenofibrate analogs include those defined in U.S. Pat. No. 4,800,079. By way of example, gemfibrozil could be used in the methods disclosed herein.

Fenofibrate is optionally dissolved in a proper solvent or solubilizers. Fenofibrate is known to be soluble in many different solubilizers, including, for example, anionic (e.g. SDS) and non-ionic (e.g. Triton X-100) surfactants, complexing agents (N-methyl pyrrolidone). Liquid and semi-solid formulations with improved bioavailability for oral administration of fenofibrate or fenofibrate derivatives are described in International Patent Application Publication No. WO 2004/002458, which is incorporated herein by reference in its entirety.

Kaempferol

Kaempferol (3, 5, 7-trihydroxy-2-(4-hydroxyphenyl)-4H-1-benzopyran-4-one), a naturally occurring flavonoid found in many edible plants (e.g., tea, broccoli, cabbage, kale, beans, endive, leek, tomato, strawberries and grapes) and possesses a range of pharmacological features, including antioxidant, anti-inflammatory, neuroprotective, anti-atherogenic, and anticancer properties [19, 20].

Evidence from in vitro and in vivo investigations suggests that kaempferol might provide P as a therapeutic candidate for Alzheimer's disease (AD). Kaempferol prevents β-amyloid-induced toxicity and aggregation effects in vitro within mouse cortical neurons, PC12 neuroblastoma and T47D human breast cancer cells [21-23]. Likewise, a flavonol mixture from Ginkgo leaves, containing quercetin, kaempferol and isorhamnetin, stimulated the BDNF signaling pathway and reduced β-amyloid accumulation within neurons isolated from a double transgenic AD mouse model (TgAPPswe/PS1e9). In vivo studies in these double transgenic AD mice confirmed enhanced BDNF expression following flavonol administration, correlating with improved cognitive function [24]. Kaempferol was also noted to inhibit oxidative stress, elevate superoxide dismutase (SOD) activity in the hippocampus, and improve learning and memory capabilities in mice with D-galactose-induced memory impairment [25]. Pre-treatment with kaempferol or products containing kaempferol provide protection against dopaminergic neurotoxicity within MPTP, 6-OHDA, or rotenone neurotoxicant animal models of PD [26-29].

Telmisartan

“Sartans,” for example, valsartan and telmisartan have been shown to confer neuroprotective and anti-inflammatory effects in animal models of retinal angiogenesis and neovascularization (Kurihara et al., Investigative Ophthalmology & Visual Science. 2006; 47(12):5545-5552; Nagai et al., Investigative Ophthalmology & Visual Science. 2007; 48(9):4342-4350; Sugiyama et al., Experimental Eye Research. 2007; 85(3):406-412; Wilkinson-Berka et al., American Journal of Hypertension. 2007; 20(4):423-430 and Phipps et al., Investigative Ophthalmology & Visual Science. 2007; 48(2):927-934).

Pharmaceutical Compositions and Routes of Administration

In some embodiments, the fenofibrate or analog thereof (and optionally an esterase inhibitor) are formulated into one or more compositions with a suitable carrier, excipient or diluent. In some embodiments, the fenofibrate or analog thereof and esterase inhibitor (e.g., kempferol or telmisartan) are formulated into the same composition. In alternative embodiments, the fenofibrate or analog thereof and esterase inhibitor (e.g., kaempferol or telmisartan) are formulated into separate compositions. In some embodiments, the fenofibrate or analog thereof and esterase inhibitor are administered concomitantly (optionally in the same or different compositions). In some embodiments, the fenofibrate or analog thereof and esterase inhibitor are administered sequentially.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Carrier encompasses any excipient, diluent, filler, salt, buffer, stabilizer, solubilizer, lipid, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Depending on the intended mode of administration, the pharmaceutical composition can be in the form of solid, semi-solid, or liquid dosage forms, such as, for example, tablets, suppositories, pills, capsules, powders, liquids, aerosols, or suspensions, preferably in unit dosage form suitable for single administration of a precise dosage. The compositions will include a therapeutically effective amount of the compound(s) described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained. Compositions containing fenofibrate or analog thereof and/or esterase inhibitor (e.g., kaempferol or telmisartan) described herein or pharmaceutically acceptable salts or prodrugs thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Compositions described herein can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compounds described herein or pharmaceutically acceptable salts or prodrugs thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of fenofibrate or analog thereof (and optionally in combination with an esterase inhibitor, e.g., kaempferol or telmisartan) or pharmaceutically acceptable salts or prodrugs thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

A pharmaceutical composition is typically formulated to be compatible with its intended route of administration, e.g., ocular, oral, mucosal, topical, transdermal, or parenteral. Supplementary active compounds can also be incorporated into the compositions.

In some embodiments, a composition comprising fenofibrate and/or esterase inhibitor (e.g., kaempferol or telmisartan) is formulated for delivery to the eye of a subject (e.g., subconjunctivally, retrobulbarly, periocularly, subretinally, suprachoroidally, or intraocularly). Suitable ophthalmic carriers are known to those skilled in the art and all such conventional carriers may be employed. Exemplary compounds incorporated to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylannmonium bromide, polaxamers, spans, tweens, bile salts and lecithin), terpene (d-limonene, alphaterpeneol, 1,8-cineole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist. Thus, a particular carrier may take the form of a sterile, ophthalmic ointment, cream, gel, solution, or dispersion. Also including as suitable ophthalmic carriers are slow release polymers, e.g., “Ocusert” polymers, “Hydron” polymers, etc.

Exemplary ophthalmic viscosity enhancers that can be used in the present formulation include: carboxymethyl cellulose sodium; methylcellulose; hydroxypropyl cellulose; hydroxypropylmethyl cellulose; hydroxyethyl cellulose; polyethylene glycol 300; polyethylene glycol 400; polyvinyl alcohol; and providone.

Some natural products, such as veegum, alginates, xanthan gum, gelatin, acacia and tragacanth, may also be used to increase the viscosity of ophthalmic solutions.

A tonicity is important because hypotonic eye drops cause an edema of the cornea, and hypertonic eye drops cause deformation of the cornea. The ideal tonicity is approximately 300 mOsM. The tonicity can be achieved by methods described in Remington: The Science and Practice of Pharmacy, known to those versed in the art.

The customary adult fenofibrate dosage is three gelatin capsules per day, each containing 100 mg of fenofibrate. One of skill in the art can select a dosage or dosing regimen by selecting an effective amount of the fenofibrate. Such an effective amount includes an amount that induces PGC-1α expression in RPE cells, an amount that has anti-inflammatory properties, an amount that reduces one or more effects of oxidative stress. It is contemplated that administration of fenofibrate or analog thereof (and optionally in combination with kaempferol or telmisartan) in combination will reduce the effective dose of fenofibrate or analog thereof necessary in a subject compared to administration of fenofibrate or analog thereof alone.

Optionally, the fenofibrate or analog thereof and kaempferol or telmisartan is administered daily.

The term “effective amount”, as used herein, is defined as any amount sufficient to produce a desired physiologic response. By way of example, the systemic dosage of the fenofibrate or analog thereof and kaempferol or telmisartan can be 1-1000 mg daily, including for example, 300 to 400 mg daily (administered for example in 1-5 doses). One of skill in the art would adjust the dosage as described below based on specific characteristics of the inhibitor, the subject receiving it, the mode of administration, type and severity of the disease to be treated or prevented, and the like. Furthermore, the duration of treatment can be for days, weeks, months, years, or for the life span of the subject. For example, administration to a subject with or at risk of developing a neurodegenerative disease could be at least daily (e.g., once, twice, three times per day), every other day, twice per week, weekly, every two weeks, every three weeks, every 4 weeks, every 6 weeks, every 2 months, every 3 months, or every 6 months, for weeks, months, or years so long as the effect is sustained and side effects are manageable.

Effective amounts and schedules for administering fenofibrate or analog thereof and kaempferol or telmisartan can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, cell death, and the like. Generally, the dosage will vary with the type of neurodegenerative disease, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily.

Combination Therapy

In some embodiments, the methods described herein further comprise administering another therapeutic for the treatment of age-related macular degeneration (AMD). In some embodiments, the additional therapeutic includes, but is not limited to, is Lucentis™ (ranibizumab), Avastin™′(bevacizumab), Eylea™ (aflibercept) or Macugen™ (pegaptanib), photodynamic therapy, laser treatment or combinations thereof.

In some embodiments, the combination therapy employing fenofibrate or analog thereof and an esterase inhibitor, e.g., kaempferol or telmisartan, described herein may precede or follow administration of additional therapeutic(s) by intervals ranging from minutes to weeks to months. For example, separate modalities are administered within about 24 hours of each other, e.g., within about 6-12 hours of each other, or within about 1-2 hours of each other, or within about 10-30 minutes of each other. In some situations, it may be desirable to extend the time period for treatment significantly, where several days (2, 3, 4, 5, 6 or 7 days) to several weeks (1, 2, 3, 4, 5, 6, 7 or 8 weeks) lapse between the respective administrations of different modalities. Repeated treatments with one or both agents/therapies of the combination therapy is specifically contemplated.

Monitoring Efficacy of Therapy

Methods for measuring PGC-1α induction and activity are known in the art and are provided in Example 2 below. See, for example, Ruiz et al. (2012) A cardiac-specific robotized cellular assay identified families of human ligands as inducers of PGC-1α expression and mitochondrial biogenesis PLoS One: 7: e46753. PGC-1α levels can be assessed directly using, for example, an antibody to PGC-1α or other means of detection. PGC-1α activity can be detected including by way of example by assessing modulation of mitochondrial function, e.g., oxidative metabolism and can be assessed by detecting the activity or expression of a mitochondrial gene, e.g., LDH-2, ATP5j, or the like.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

### Example 1

Materials and Methods:

Human Subjects: The Institutional review board of the University of California Irvine approved research with human subjects (Approval #2003-3131). All participating subjects provided informed consent and clinical investigations were performed according to the tenets of Declaration of Helsinki.

Cell culture: Passage 5 AMD ARPE-19 transmitochondrial cybrid cells were created as described previously (72). Briefly, these cybrid cells were prepared by polyethylene glycol fusion of mitochondrial DNA-deficient APRE-19 (Rho0) cell line with platelets isolated from AMD patients. All cybrids used in this study belonged to the ‘H’ mitochondrial DNA haplogroup.

Treatment with fenofibrate and Esterase Inhibitors (kaempferol and telmisartan): fenofibrate stock solution of 40 mM concentration was prepared at 15 mg/mL in DMSO. fenofibrate stock was diluted in culture media to obtain a working concentration of 50 μM which was used for all experiments in this study. Stock solutions of 20 mM kaempferol and 10 mM telmisartan were prepared in DMSO and were diluted in culture media to obtain the following working concentrations: kaempferol at 5 μM, 10 μM, and 20 μM; telmisartan at 2.5 μM, 5 μM, and 10 μM.

Quantitative Real-Time PCR: RNA extraction, cDNA synthesis, and qRT-PCR analysis were performed as described previously (73). QuantiTect Primer Assays were used to study the expression of Caspase-3 gene (Cat. #QT00023947, Qiagen, Germantown, Md.), BAX gene (Cat. #QT00031192, Qiagen), HIF1α gene (Cat. #QT00083664, Qiagen), CFH gene (Cat. #QT00001624, Qiagen), and SOD2 gene (Cat. #QT01008693, Qiagen). KiCqStart® SYBR® green primers were used to examine the expression of PGC-1a, NRF-1, NRF-2, PPAR-α, PPAR-γ, VEGF, IL-18, and IFNB1 genes (Cat. #kspq12012, Sigma, St. Louis, Mo.). Specific housekeeper genes used were HPRT1 (Cat. #QT00059066, Qiagen), ALAS variant 1 (Cat. #QT01160467, Qiagen), and HMBS (Cat. #QT00014462). TaqMan gene expression master mix (Cat. #4369016, Life Technologies) and TaqMan gene expression assays were used to examine the expression of the MT-RNR2 gene (Assay ID: Hs02596860_s1, Life Technologies), for which GAPDH (Assay ID: Hs02786624_g1, Life Technologies) was used as a housekeeper gene. Data analysis was performed using ΔΔCt method which was calculated by subtracting ΔCt of the AMD group from ΔCt of the normal group. ΔCt was the difference between the Cts (threshold cycles) of the target gene and Cts of the housekeeper gene (reference gene). Fold change was calculated using the following formula: Fold change=2^{ΔΔct}.

Cell viability assay (MTT assay): The numbers of viable cells were measured using the MTT (3-(4,5-dimethylthiazol-2-yl)-2,5-diphenyltetrazolium bromide) assay. Cells were plated in 96-well tissue culture plates, treated with 50 μM fenofibrate followed by addition of MTT. Cells were incubated at 37° C. for 1 h, followed by addition of DMSO. Absorbance was measured at 570 nm and background absorbance measured at 630 nm. Normalized absorbance values were obtained by subtracting background absorbance from signal absorbance. The colorimetric signal obtained was proportional to the cell number.

### Example 2—Fenofibrate Positively Regulated the Mitochondrial Biogenesis Pathway in AMD RPE Cells

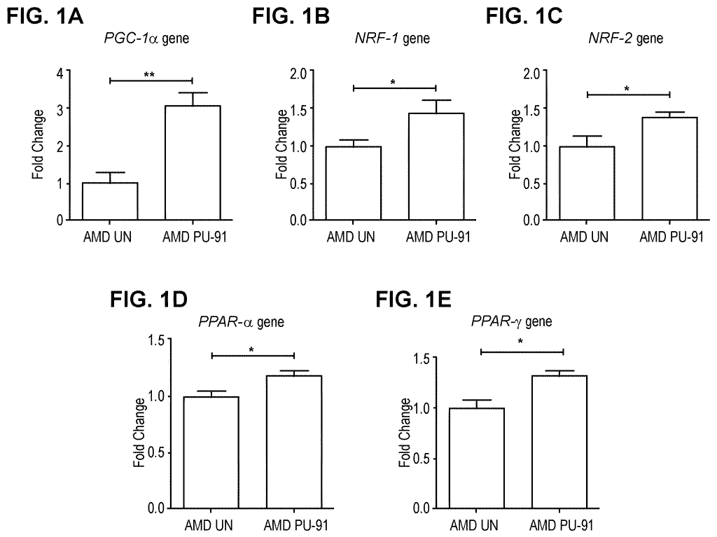

As shown in FIG. 1, fenofibrate significantly upregulated the gene expression of PGC-1α by 208% (P=0.0018; AMD UN: 1±0.29, n=5; AMD fenofibrate: 3.08±0.35, n=5) (FIG. 1A), NRF-1 by 46% (P=0.04; AMD UN: 1±0.08, n=4; AMD fenofibrate: 1.46±0.16, n=4) (FIG. 1B), NRF-2 by 38% (P=0.04; AMD UN: 1±0.13, n=5; AMD fenofibrate: 1.38±0.08, n=5) (FIG. 1C), PPAR-α by 19% (P=0.02; AMD UN: 1±0.05, n=5; AMD fenofibrate: 1.19±0.05, n=5) (FIG. 1D), and PPAR-γ by 32% (P=0.03; AMD UN: 1±0.09, n=5; AMD fenofibrate: 1.32±0.08, n=5) (FIG. 1E) in AMD cells compared to their untreated counterparts. By modulating the expression mitochondrial biogenesis markers, fenofibrate plays a critical role in mitochondrial and cellular health.

### Example 3—Fenofibrate Improved Mitochondrial Function in AMD RPE Cells

The following experiments were performed to compare the mitochondrial membrane potential (ΔΨm) between untreated and fenofibrate-treated AMD cybrids using the JC-1 and MitoSOX assays.

Mitochondrial membrane potential (JC-1) assay: The JC-1 assay uses a unique cationic dye i.e., 5,5′,6,6′-tetrachloro-1,1′,3,3′-tetraethylbenzimidazolylcarbocyanine iodide, to detect loss of mitochondrial membrane potential. JC-1 1× reagent was prepared by diluting 100× JC-1 reagent in assay buffer to 1:100 dilutions. AMD cybrids were plated in 24-well tissue culture plates for 24 hr followed by treatment with 50 μM fenofibrate. 1× JC-1 reagent was added to cells and incubated for 15 min at 37° C. JC-1 reagent in the wells was then replaced with DPBS and fluorescence was measured as follows: Red fluorescence (Live cells): Excitation 550 nm and Emission 600 nm; Green fluorescence (Apoptotic cells): Excitation 485 nm and Emission 535 nm. Ratio of Red/Green was used for analysis. Lower ratio corresponded to higher apoptotic/dead cell number.

MitoSOX assay: The fluorogenic MitoSOX Red dye (Cat. #M36008, Invitrogen, Grand Island, N.Y., USA) is a live-cell permeant reagent that detects mitochondrial superoxide in cells. MitoSOX Red reagent oxidized by superoxide has red fluorescence that can be quantified. AMD cybrids were plated in 24-well tissue culture plates. Stock solution of 5 mM MitoSOX reagent was diluted with HBSS (Hank's balanced salt solution) buffer to obtain a 5 μM working solution. Cells were treated with 5 μM MitoSOX reagent and incubated for 10 min at 37° C. Cells were then washed with HBSS buffer, and fluorescence was measured at excitation/emission maxima of 510/580 nm.

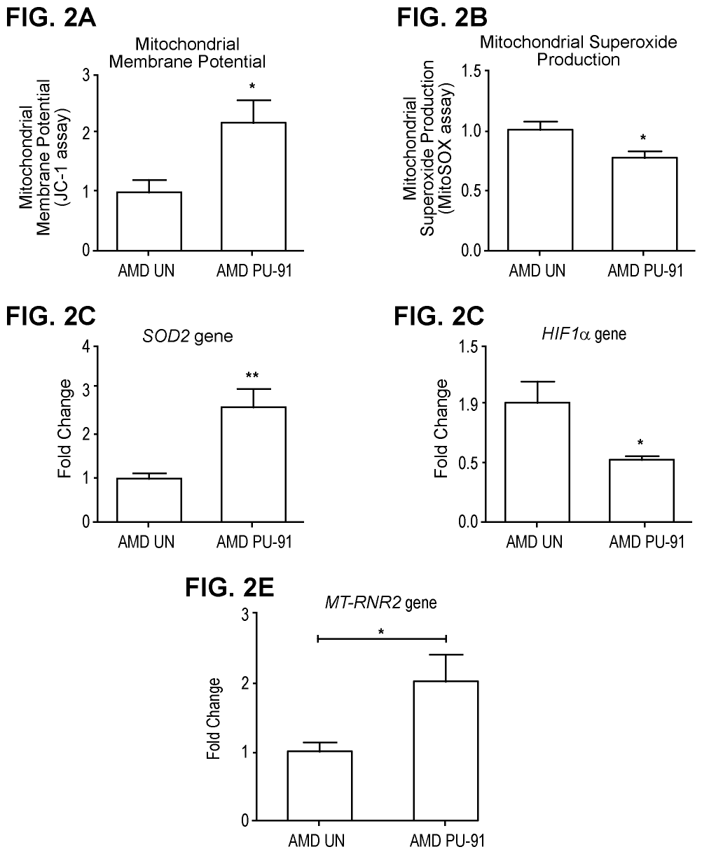

As shown in FIG. 2, fenofibrate-treated AMD cybrid cells had increased mitochondrial membrane potential (JC-1 assay) (116%) (P=0.04; AMD UN: 1±0.09, n=3; AMD fenofibrate: 2.16±0.38, n=3), (FIG. 2A), and significantly lower amount of mitochondrial superoxides (MitoSOX assay) (23%) (P=0.04; AMD UN: 1±0.06, n=3; AMD fenofibrate: 0.77±0.04, n=3), (FIG. 2B). Furthermore, fenofibrate-treated AMD cells showed up-regulation of SOD2, a mitochondrial antioxidant gene, by 160%, (P=0.0035; AMD UN: 1±0.11, n=5; AMD fenofibrate: 2.6±0.37, n=5) (FIG. 2C), and reduced gene expression of HIF1α (47%) (P=0.03; AMD UN: 1±0.19, n=3; AMD fenofibrate: 0.53±0.02, n=4) (FIG. 2D).

Fenofibrate up-regulated MT-RNR2 (Mitochondrially Encoded 16S RNA) gene in AMD RPE cells. Treatment with fenofibrate drug caused a 104% higher expression of MT-RNR2 gene in AMD RPE cells (P=0.039; AMD UN: 1±0.15, n=5; AMD fenofibrate: 2.04±0.39 n=5) (FIG. 2E), implying that increased production of mitochondrial derived peptides (MDPs) could be one of the mechanisms via which fenofibrate rescues cells. Treatment with fenofibrate improved ΔΨm significantly in AMD cybrid cells, suggesting that fenofibrate can protect mitochondrial membrane integrity and function.

Results also demonstrated that there was significantly diminished mitochondrial superoxide production in fenofibrate-treated AMD cells compared to untreated AMD cells. It was also observed that fenofibrate mediated antioxidant effects in AMD cybrids by upregulating SOD2/MnSOD gene substantially. Hypoxic stress and activation of HIF1α has been implicated in AMD. ROS and HIF1α cause VEGF activation thereby triggering angiogenesis and subsequent choroidal neovascularization in wet AMD (60, 61). Fenofibrate-treated AMD cybrids had lower expression of HIF1α gene, suggesting that fenofibrate exhibits hypoxia-suppressing effects. Cumulatively, these results highlight a key role of fenofibrate in decreasing oxidative stress in AMD cells.

### Example 4—Fenofibrate Prevented Cell Death in AMD RPE Cells

The following experiment was performed to assess the expression of caspase-3 and BAX in RPE cells, both of which are markers of cell apoptosis (62).

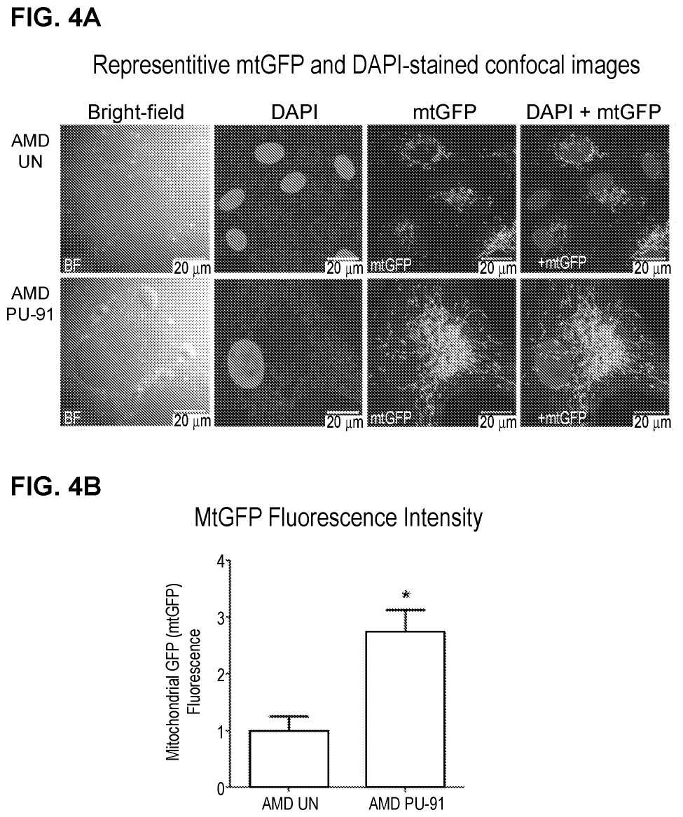

Results indicated that higher number of viable cells and downregulation of Caspase-3 and BAX genes was observed in fenofibrate-treated AMD cells, indicating that fenofibrate prevents mitochondria-induced apoptotic cell death in AMD transmitochondrial RPE cells. As shown in FIG. 4, significantly higher cell viability (51%) and increased expression of Caspase-3 gene (34%) (P=0.02; AMD UN: 1±0.12, n=5; AMD fenofibrate: 0.66±0.03, n=5) (FIG. 4A) and BAX gene (21%) (P=0.01; AMD UN: 1±0.05, n=5; AMD fenofibrate: 0.79±0.03, n=5) (FIG. 4B) was observed in fenofibrate-treated AMD cells compared to their untreated counterparts.

### Example 5—Fenofibrate Enhanced Mitochondrial GFP (mtGFP) Fluorescence in AMD RPE Cells

The following experiment was performed to compare mitochondrial density between untreated and fenofibrate-treated AMD cells.

CellLight Mitochondrial GFP staining and Confocal microscopy: Staining with CellLight Mitochondrial GFP probe (Cat. #C10600, Thermo Fisher Scientific, MA, USA) and confocal microscopy were performed as described previously (74). Cells were plated in 4-well tissue culture chamber slides, stained with CellLight mtGFP for 24 hr and incubated overnight at 37° C. The cells were washed with 1×TBS (Tris buffered saline), fixed in paraformaldehyde and mounted in DAPI. Confocal z-stack images were captured using the LSM-700 Confocal microscope (Zeiss, Thornwood, N.Y., USA). Images were quantified using ZEN 2 lite software (Zeiss).

RPE cells were transduced with CellLight reagent which is a GFP-E1 alpha pyruvate dehydrogenase leader peptide construct with a mammalian promoter. This fluorescent baculoviral fusion construct provides precise targeting to mitochondria. Herein, treatment with fenofibrate enhanced mitochondrial GFP fluorescence appreciably in AMD cells compared to their untreated counterparts, indicating that fenofibrate can prevent mitochondrial loss in AMD cells. These results are consistent with a previous study wherein Humanin G, a mitochondrial-derived peptide, rescued AMD mitochondria in RPE cybrid cells.ⁱ

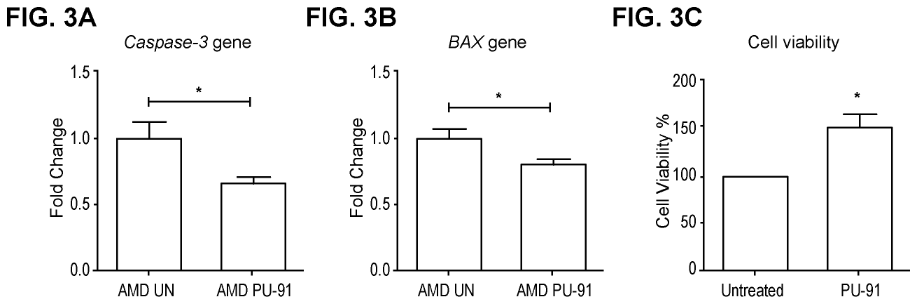

FIG. 3A shows representative confocal images of AMD RPE cells stained with DAPI (blue) and mitochondrial GFP stain (green). Panel 1 shows bright-field images, panel 2 shows DAPI (blue)-stained images, panel 3 shows mtGFP (green)-stained images, and panel 4 shows merge (DAPI+mtGFP) images. Fenofibrate-treated AMD cells showed an increase in mtGFP fluorescence intensity by 173% (P=0.0397; AMD UN: 1±0.25, n=3; AMD fenofibrate: 2.73±0.39, n=3) (FIG. 3B) compared to the untreated AMD cells.

### Example 6—Fenofibrate Regulated Inflammation and Complement in AMD RPE Cells

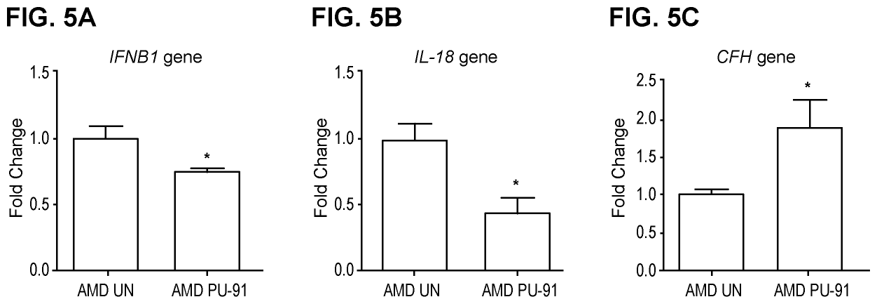

Treatment with fenofibrate altered the gene expression of inflammatory markers, IFNB1 (25% decrease) (P=0.019; AMD UN: 1±0.08, n=4; AMD fenofibrate: 0.75±0.02, n=4) (FIG. 5A), IL-18 (56% decrease) (P=0.02; AMD UN: 1±0.13, n=4; AMD fenofibrate: 0.44±0.12, n=4) (FIG. 5B), and of a complement inhibitor CFH (88% increase) (P=0.04; AMD UN: 1±0.07, n=4; AMD fenofibrate: 1.88±0.38, n=3) (FIG. 5C).

### Example 7—Additive Effect of Fenofibrate and Esterase Inhibitors (Kaempferol and Telmisartan) on Cell Viability in AMD RPE Cells

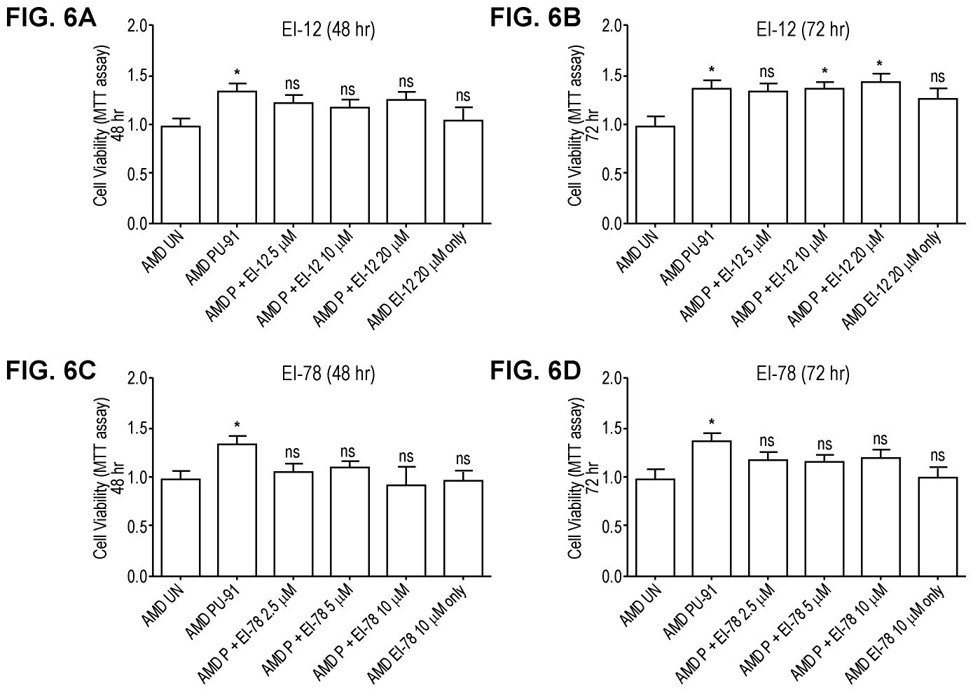

As shown in FIG. 6, Table 1 and Table 2, treatment with fenofibrate+kaempferol at varying concentrations (i.e., kaempferol 5 uM, 10 uM, and 20 uM) did not change the cell viability of AMD cells at 48 hr (FIG. 6A).

TABLE 1
Effects of fenofibrate and kaempferol on cell viability

| | | AMD | AMD | AMD | AMD | AMD |
|---|---|---|---|---|---|---|
| Kaempferol | | Only | fenofibrate + | fenofibrate + | fenofibrate + | Only |
| 48 hr Time | AMD | fenofibrate- | kaempferol | kaempferol | kaempferol | kaempferol |
| point | UN | treated | 5 μM | 10 μM | 20 μM | 20 μM |
| Mean ± SEM | 1 ± 0.07 | 1.33 ± 0.09 | 1.215 ± 0.09027 | 1.176 ± 0.07125 | 1.253 ± 0.07453 | 1.037 ± 0.1413 |
| | | AMD | AMD | AMD | AMD | AMD |
| Kaempferol | | Only | fenofibrate + | fenofibrate + | fenofibrate + | Only |
| 72 hr Time | AMD | fenoibrate- | kaempferol | kaempferol | kaempferol | kempferol |
| point | UN | treated | 5 μM | 10 μM | 20 μM | 20 μM |
| Mean ± SEM | 1 ± 0.09 | 1.37 ± 0.07 | 1.335 ± 0.08792 | 1.361 ± 0.07264 | 1.452 ± 0.06857 | 1.259 ± 0.1060 |

TABLE 2
Effects of fenofibrate + telmisartan on cell viability

| | | AMD | AMD | AMD | AMD | AMD |
|---|---|---|---|---|---|---|
| Telmisartan | | Only | fenofibrate + | fenofibrate + | fenofibrate + | Only |
| 48 hr Time | AMD | fenfibrate- | telmisartan | telmisartan | telmisartan | telmisartan |
| point | UN | treated | 2.5 μM | 5 μM | 10 μM | 10 μM |
| Mean ± SEM | 1 ± 0.07 | 1.33 ± 0.09 | 1.05 ± 0.09 | 1.10 ± 0.06 | 0.91 ± 0.19 | 0.95 ± 0.11 |
| | | AMD | AMD | AMD | AMD | AMD |
| Telmisartan | | Only | fenofibrate + | fenofibrate + | fenofibrate + | Only |
| 72 hr Time | AMD | fenfibrate- | telmisartan | telmisartan | telmisartan | telmisartan |
| point | UN | treated | 2.5 μM | 5 μM | 10 μM | 10 μM |
| Mean ± SEM | 1 ± 0.09 | 1.37 ± 0.07 | 1.19 ± 0.04 | 1.17 ± 0.08 | 1.21 ± 0.09 | 0.99 ± 0.09 |

By contrast, a significant difference in cell viability was observed between the untreated AMD group and the group treated with fenofibrate+kaempferol 10 uM and fenofibrate+kaempferol 20 uM at the 72 hr time point (FIG. 6B). The AMD cells treated with kaempferol 20 uM only did not show any difference in cell viability compared to untreated AMD group. Compared to untreated AMD cells, treatment with fenofibrate+telmisartan at varying concentrations (i.e., telmisartan 2.5 uM, 5 uM, and 10 uM) to fenofibrate eliminated the cell viability cytoprotection of fenofibrate alone on AMD RPE cells at 48 hr (FIG. 6C) and 72 hr (FIG. 6D) time points.

No significant changes in cell viability were observed between AMD cells treated with only fenofibrate and those treated with fenofibrate+EI12 or fenofibrate+telmisartan.

### Example 8—Additive Effect of Fenofibrate and Esterase Inhibitors (Kaempferol and Telmisartan) on Gene Expression in AMD RPE Cells

Treatment with fenofibrate+kaempferol/telmisartan at different concentrations (i.e., kaempferol: 5 uM, 10 uM, and 20 uM; telmisartan: 2.5 uM, 5 uM, and 10 uM) altered the expression of PGC-1a, Caspase-3, IL-18, VEGF, SOD2 genes in AMD RPE cells at the 72 hr time point.

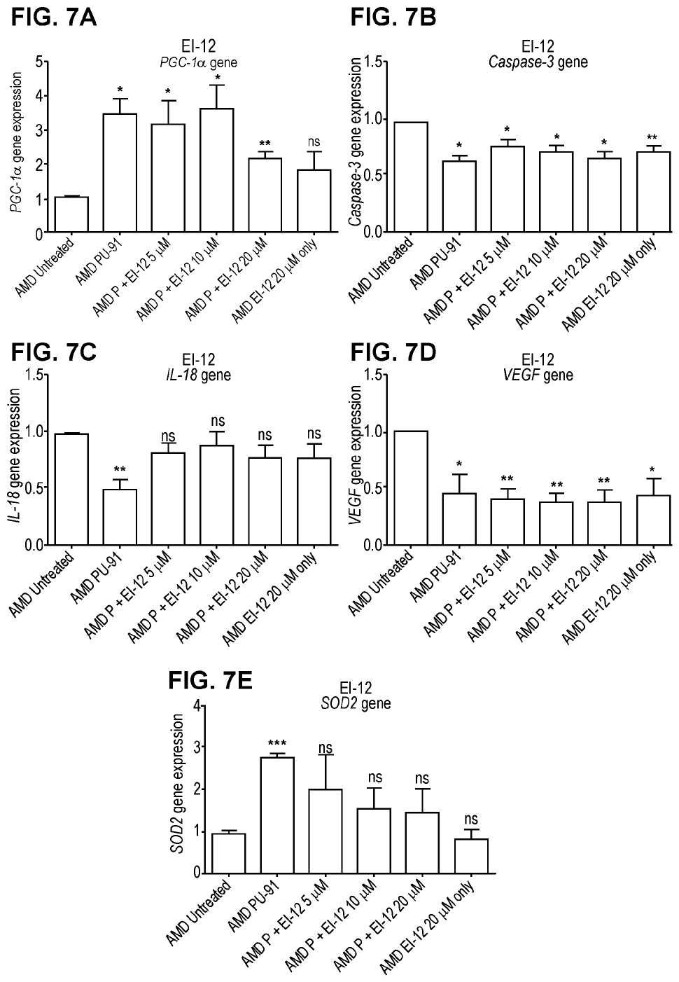

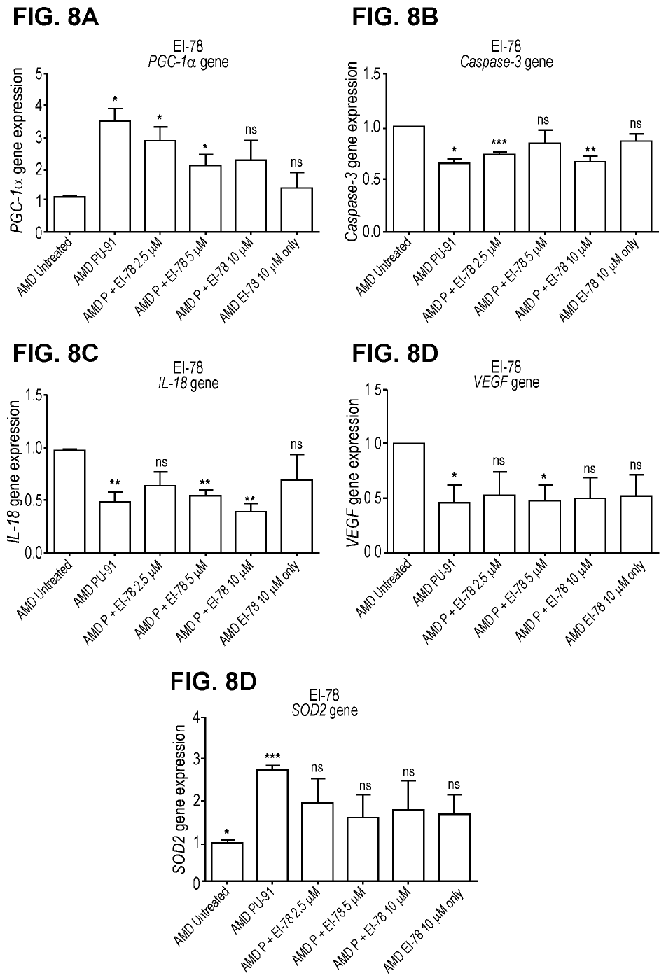

PGC-1α: kaempferol—Compared to untreated AMD cybrids, significant PGC-1α upregulation was observed in fenofibrate-treated, F+kaempferol 5 μM (216%), F+kaempferol 10 μM (263%), and F+kaempferol 20 μM (115%) groups (FIG. 7A) (Table 3 below). telmisartan—Compared to untreated AMD cybrids, significant PGC-1α upregulation was observed in fenofibrate-treated, F+telmisartan 2.5 μM (189%), and F+telmisartan 5 μM (109%) groups (FIG. 8A) (Table 4 below).

Caspase-3: kaempferol—Compared to untreated AMD cybrids, significant Caspase-3 downregulation was observed in fenofibrate-treated, F+kaempferol 5 μM (22%), F+kaempferol 10 μM (27%), F+kaempferol 20 μM (34%), and only kaempferol 20 μM (26%) groups (FIG. 7B) (Table 3). telmisartan—Compared to untreated AMD cybrids, significant Caspase-3 downregulation was observed in fenofibrate-treated, F+telmisartan 2.5 μM (26%), and F+telmisartan 10 μM (34%) groups (FIG. 8B) (Table 4 below).

IL-18: kaempferol—Compared to untreated AMD cybrids, significant IL-18 downregulation was observed only in the fenofibrate-treated group (FIG. 7C) (Table 3). telmisartan—Compared to untreated AMD cybrids, significant IL-18 downregulation was observed in fenofibrate-treated, F+telmisartan 5 μM (45%), and F+telmisartan 10 μM (61%) groups (FIG. 8C) (Table 4 below).

VEGF: kaempferol—Compared to untreated AMD cybrids, significant VEGF downregulation was observed in fenofibrate-treated, F+kaempferol 5 μM (60%), F+kaempferol 10 μM (63%), F+kaempferol 20 μM (63%), and only kaempferol 20 μM (58%) groups (FIG. 7D) (Table 3). telmisartan—Compared to untreated AMD cybrids, significant VEGF downregulation was observed in fenofibrate-treated and F+telmisartan 5 μM (53%) groups (FIG. 8D) (Table 4 below).

SOD2: kaempferol—Compared to untreated AMD cybrids, significant SOD2 upregulation was observed only in the fenofibrate-treated group (FIG. 7E) (Table 3 below). telmisartan—Compared to untreated AMD cybrids, significant SOD2 upregulation was observed only in the fenofibrate-treated group (FIG. 8E) (Table 4 below).

TABLE 3
Effects of fenofibrate and kaempferol on gene expression

| | | | AMD | AMD | AMD | AMD | AMD |
|---|---|---|---|---|---|---|---|
| | | | Only | Fenofibrate + | Fenofibrate + | Fenofibrate + | Only |
| | | | fenofibrate- | kaempferol | kaempferol | kaempferol | kaempferol |
| Kaempferol | | AMD UN | treated | 5 μM | 10 μM | 20 μM | 20 μM |
| PGC-1a | Mean ± | 1 ± 0.02 | 3.48 ± 0.45 | 3.16 ± 0.71 | 3.63 ± 0.69 | 2.15 ± 0.24 | 1.82 ± 0.57 |
| | SEM | | | | | | |
| Caspase-3 | Mean ± | 1 ± 0.0002 | 0.65 ± 0.04 | 0.78 ± 0.06 | 0.73 ± 0.06 | 0.66 ± 0.09 | 0.74 ± 0.045 |
| | SEM | | | | | | |
| IL-18 | Mean ± | 1 ± 0.004 | 0.49 ± 0.09 | 0.82 ± 0.09 | 0.88 ± 0.12 | 0.77 ± 0.11 | 0.78 ± 0.13 |
| | SEM | | | | | | |
| VEGF | Mean ± | 1 ± 0.002 | 0.45 ± 0.17 | 0.40 ± 0.09 | 0.37 ± 0.08 | 0.37 ± 0.12 | 0.42 ± 0.16 |
| | SEM | | | | | | |
| SOD2 | Mean ± | 1 ± 0.06 | 2.81 ± 0.11 | 2.05 ± 0.83 | 1.57 ± 0.53 | 1.51 ± 0.57 | 0.88 ± 0.22 |
| | SEM | | | | | | |

TABLE 4
Effects of fenofibrate and telmisartan on gene expression

| | | | AMD | AMD | AMD | AMD | AMD |
|---|---|---|---|---|---|---|---|
| | | | Only | fenofibrate + | fenofibrate + | fenofibrate + | Only |
| | | | fenofibrate- | telmisartan | telmisartan | telmisartan | telmisartan |
| telmisartan | | AMD UN | treated | 2.5 μM | 5 μM | 10 μM | 10 μM |
| PGC-1a | Mean ± | 1 ± 0.02 | 3.48 ± 0.45 | 2.89 ± 0.41 | 2.09 ± 0.37 | 2.26 ± 0.64 | 1.40 ± 0.48 |
| | SEM | | | | | | |
| Caspase-3 | Mean ± | 1 ± 0.0002 | 0.65 ± 0.04 | 0.74 ± 0.019 | 0.85 ± 0.12 | 0.66 ± 0.05 | 0.86 ± 0.06 |
| | SEM | | | | | | |
| IL-18 | Mean ± | 1 ± 0.004 | 0.49 ± 0.09 | 0.65 ± 0.13 | 0.55 ± 0.06 | 0.39 ± 0.08 | 0.71 ± 0.25 |
| | SEM | | | | | | |
| VEGF | Mean ± | 1 ± 0.002 | 0.45 ± 0.17 | 0.53 ± 0.21 | 0.47 ± 0.14 | 0.51 ± 0.18 | 0.52 ± 0.19 |
| | SEM | | | | | | |
| SOD2 | Mean ± | 1 ± 0.06 | 2.81 ± 0.11 | 1.99 ± 0.59 | 1.62 ± 0.57 | 1.82 ± 0.69 | 1.71 ± 0.46 |
| | SEM | | | | | | |

Discussion:

Mitochondrial stabilization and protection may be a potential mechanism by which fenofibrate protects AMD RPE cybrid cells. To compare mitochondrial density between untreated and fenofibrate-treated AMD cybrid cells, cells were transduced with CellLight reagent which is a GFP-E1 alpha pyruvate dehydrogenase leader peptide construct with a mammalian promoter. This fluorescent baculoviral fusion construct provides precise targeting to mitochondria. Herein, treatment with fenofibrate enhanced mitochondrial GFP fluorescence appreciably in AMD cybrid cells compared to their untreated counterparts, indicating that fenofibrate can prevent mitochondrial loss in AMD cells. These results are consistent with a previous study wherein Humanin G, a mitochondrial-derived peptide, rescued AMD mitochondria in RPE cybrid cells (67).

Fenofibrate attenuated IL-18 gene expression, thereby reducing mtDNA damage-induced inflammation in AMD cybrid cells. This is significant because elevation of pro-inflammatory cytokines in the serum and ocular fluids of AMD patients has been reported. Ijima et al suggested association of IL-18 with dry AMD since patients with dry AMD had higher IL-18 serum levels; this study also demonstrated IL-18-induced RPE cell degeneration in mouse eye (68). AMD cells treated with fenofibrate showed reduced expression of IFNB1 gene which has been demonstrated to reduce human RPE cell proliferation (69). As shown previously, AMD cybrids have decreased expression of CFH, an inhibitor of complement pathway, indicating activation of complement in AMD cells (70). Moreover, AMD patients carrying the high-risk allele for CFH showed substantial retinal mtDNA damage (71). Significant increase in CFH gene expression was observed in fenofibrate-treated AMD cybrids, suggesting inhibition of complement by fenofibrate.

Next, the effects of co-administration of fenofibrate with esterase inhibitors (EI)—kaempferol and telmisartan was investigated. Administration of fenofibrate in humans/animals results in a large first pass effect, converting the vast majority of fenofibrate to its primary metabolite, fenofibrate*, which is inactive as a PGC-1α upregulator. We identified the mechanism of fenofibrate→fenofibrate* conversion and identified two esterase inhibitors—kaempferol and telmisartan, that when co-administered with fenofibrate largely block conversion to fenofibrate*, thereby markedly increasing bioavailability of fenofibrate. The following co-administration combinations were tested—

1) fenofibrate (50 μM)+kaempferol (5 μM),
2) fenofibrate (50 μM)+kaempferol (10 μM),
3) fenofibrate (50 μM)+kaempferol (20 μM),
4) fenofibrate (50 μM)+telmisartan (2.5 μM),
5) fenofibrate (50 μM)+telmisartan (5 μM),
6) fenofibrate (50 μM)+telmisartan (10 μM),
7) kaempferol (20 μM),
8) telmisartan (10 μM),
9) fenofibrate (50 μM), and
10) AMD untreated.

No drastic change either in cell viability or gene expression (of PGC-1a, Caspase-3, IL-18, VEGF, SOD2) was observed when treated with a combination of fenofibrate+kaempferol/telmisartan compared to treatment with fenofibrate alone.

To summarize, in the in vitro AMD RPE transmitochondrial cybrid cells, the fenofibrate drug: 1) regulated the mitochondrial biogenesis pathway, 2) improved mitochondrial function, 3) enhanced mitochondrial GFP fluorescence, 4) prevented apoptotic cell death, 5) regulated inflammation and complement, 6) regulated the MDP-coding MT-RNR2 gene, 7) when co-administered with kaempferol/fenofibrate, did not modulate either the viable cell count or gene expression (of PGC-1a, Caspase-3, IL-18, VEGF, SOD2) substantially compared to treatment with fenofibrate alone.

In conclusion, fenofibrate rescues AMD RPE cybrid cells, and could be used as a repositioned FDA-approved drug to prevent/treat AMD. This is a novel study that identified fenofibrate as a therapeutic target for treatment of AMD. Since it improves mitochondrial function and has already been FDA-approved, the candidate therapeutic, fenofibrate, will be a more effective treatment option for AMD than any of the available alternatives. Furthermore, it would save a substantial sum of money and time that goes into the rigorous process of FDA-approval of drugs.

### REFERENCES

1. Modenese et al., Int Arch Occup Environ Health. 2018. doi: 10.1007/s00420-018-1355-y.
2. Villegas et al., Expert Opin Drug Deliv. 2017; 14(2):273-282.
3. Krishnadev et al., Curr Opin Ophthalmol. 2010; 21(3):184-9.
4. Dib et al., Biochim Biophys Acta. 2015; 1853(11 Pt A):2897-906.
5. Jarrett et al., Ophthalmic Res. 2010; 44(3):179-90.
6. Lin et al., Invest Ophthalmol Vis Sci. 2011; 52(6):3521-9.
7. Markovets et al., PLoS One. 2011; 6(7): e21682. Epub 2011 Jul. 5.
8. Cousins, et al., Retina Today. 2015. 83-85.
9. Nashine et al., Cell Death Dis. 2017; 8(7): e2951.
10. Scarpulla, Biochim Biophys Acta. 2011; 1813(7):1269-78.
11. Lin et al., Cell Metab. 2005; 1(6):361-70.
12. Saint-Geniez et al., Am J Pathol. 2013; 182(1):255-65.
13. Iacovelli et al., Invest Ophthalmol Vis Sci. 2016; 57(3):1038-51.
14. Zheng et al., Sci Transl Med. 2010; 2(52):52ra73.
15. Handschin et al., Endocr Rev. 2006; 27(7):728-35.
16. Wang et al., Transl Neurodegener. 2016; 5:19.
17. Róna-Vörös et al., Curr Drug Targets. 2010; 11(10):1262-9.
18. Ventura-Clapier et al., Cardiovasc Res. 2008; 79(2):208-17.
19. Rao et al., Mol Cell Biochem. 2012; 367(1-2):9-18.
20. Corona et al., Neurochem Res. 2015; 40(2):308-16.
21. Ciron et al., Hum Mol Genet. 2012; 21(8):1861-76.
22. McGill et al., Cell. 2006; 127(3):465-8.
23. Johri et al., Free Radic Biol Med. 2013; 62:37-46.
24. Sweeney et al., Cell Biol. 2016; 49(1):1-6.
25. Guo et al., Am J Pathol. 2014; 184(4):1017-1029.
26. Kaarniranta et al., Int J Mol Sci. 2018; 19(8). pii: E2317.
27. Iacovelli et al., Invest Ophthalmol Vis Sci. 2016; 57(3):1038-51.
28. Jornayvaz et al., Essays Biochem. 2010; 47:69-84.
29. Hertel et al., Eur J Neurosci. 2002; 15(10):1707-11.
30. Radhakrishnan et al., Mol Cell. 2010; 38(1):17-28.
31. He M et al., PLoS One. 2014; 9(1): e84800.
32. Felszeghy et al., Redox Biol. 2018; 20:1-12.
33. Leung et al., J Biol Chem. 2003; 278(48):48021-9.
34. Virbasius et al., Genes Dev. 1993; 7(12A):2431-45.
35. Michalik et al., Pharmacol Rev. 2006; 58(4):726-41.
36. Ding et al., Am J Pathol. 2014; 184(10):2709-20.
37. Pearsall et al., BMC Biol. 2017; 15(1):113.
38. Zhu et al., Int J Ophthalmol. 2015; 8(1):52-6.
39. Carta et al., Neuroscience. 2011; 194, 250-261.
41. Escribano et al., Biochemical and Biophysical Research Communications. 2009; 379 (2), 406-410.
42. Kiaei, et al., Experimental Neurology. 2005; 191 (2), 331-336.
43. Zhu et al., PLoS One. 2013; 8(7): e68935.
44. T. Usui, et al., Investigative Ophthalmology and Visual Science. 2008; 49 (10), 4370-4376.
45. Uchiyama, et al., Molecular Vision. 2013; 19, 2135-2150.
46. Sarayba et al., Experimental Eye Research. 2005; 80 (3), 435-442.
47. Yamanaka, et al., Investigative Ophthalmology & Visual Science. 2009; 50 (1), 187-193.
48. Murata et al., Investigative Ophthalmology and Visual Science. 2000; 41 (8), 2309-2317.
49. Wigdal et al., J Neurochem. 2002; 82: 1029-1038.
50. Kroemer et al., Immunol Today 18: 44-51, 1997.
51. Lieven et al., Antioxid Redox Signal. 2003; 5(5):641-6.
52. Chong et al., Redox Biol. 2016; 9:50-56.
53. Ellis et al., Invest Ophthalmol Vis Sci. 2017; 58(5):2755-2764.
54. Nashine et al., Cell Death Dis. 2017; 8(7): e2951.
55. Seo et al., Exp Eye Res. 2012; 101:60-71.
56. Beckman et al., Trends Neurosci. 2001; 24(11 Suppl): S15-20.
57. Estévez et al., J Neurosci. 1998; 18(3):923-31.
58. Indo et al., J Clin Biochem Nutr. 2015; 56(1):1-7.
59. Justilien et al., Invest Ophthalmol Vis Sci. 2007; 48(10):4407-20.
60. Kan et al., Diagn Pathol. 2014; 9:73.
61. Schlingemann, et al., Graefes Arch. Clin. Exp. Ophthalmol. 2004; 242, 91-101.
62. Martin, et al., Graefes Arch. Clin. Exp. Ophthalmol. 2004; 242, 321-326.
63. Nashine et al., Cell Death Dis. 2017; 8(7): e2951.
64. Telegina et al., Adv Gerontol. 2016; 29(3):424-432.
65. Adler et al., Mol Vis. 1999; 5:31.
66. Okada et al., Sci Rep. 2017; 7(1):7802.
67. Nashine et al., Cell Death Dis. 2017; 8(7): e2951.
68. Nashine et al., Cell Death Dis. 2017; 8(7): e2951.
69. Ijima et al., Investigative ophthalmology & visual science. 2014; 55:6673-8.
70. Qiao et al., Ophthalmologica. 2001; 215(6):401-7.
71. Nashine et al., PLoS One. 2016; 11(8): e0159828.
72. Ferrington et al., Exp Eye Res. 2016; 145:269-277.
73. Nashine et al., Cell Death Dis. 2017; 8(7): e2951.
74. Nashine et al., Cell Death Dis. 2017; 8(7): e2951.
75. Nashine et al., Cell Death Dis. 2017; 8(7): e2951.

What is claimed is:

1. A method for treating age-related macular degeneration (AMD) in a subject in need thereof comprising administering fenofibrate to the subject.

2. The method of claim 1, wherein the AMD is dry AMD.

3. The method of claim 1 or claim 2, further comprising administering an esterase inhibitor to the subject.

4. The method of claim 3, wherein the esterase inhibitor is kaempferol or telmisartan.

5. The method of claim 4, wherein the esterase inhibitor is kaempferol.

6. The method of claim 5, wherein the fenofibrate and esterase inhibitor are administered concomitantly.

7. The method of claim 5, wherein the fenofibrate and esterase inhibitor are administered sequentially.

8. The method of any one of claims 1-7, further comprising determining that the subject has a reduced level of PGC-1α expression as compared to a control subject.

9. A method of decreasing inflammation in a retinal pigment epithelium (RPE) cybrid cell in a subject in need thereof, comprising administering fenofribrate to the subject.

10. The method of claim 7, wherein the subject is suffering from age-related macular degeneration (AMD).

11. The method of claim 9 or claim 10, wherein the AMD is dry AMD.

12. The method of claim 9, further comprising administering an esterase inhibitor to the subject.

13. The method of claim 12, wherein the esterase inhibitor is kaempferol or telmisartan.

14. The method of claim 13, wherein the esterase inhibitor is kaempferol.

15. The method of claim 12, wherein the fenofibrate and esterase inhibitor are administered concomitantly.

16. The method of claim 12, wherein the fenofibrate and esterase inhibitor are administered sequentially.

17. A method of inducing PGC-1α expression in a retinal pigment epithelium (RPE) cybrid cell comprising contacting the cell with fenofibrate.

18. A method of increasing mitochondrial content in a retinal pigment epithelium (RPE) cybrid cell comprising contacting the cell with fenofibrate.

19. The method of claim 17 or claim 18, further comprising contacting the cell with an esterase inhibitor.

20. The method of claim 19, wherein the esterase inhibitor is kaempferol or telmisartan.

21. The method of any one of claims 17-20, wherein the contacting step occurs in vivo.

22. The method of claim 20, wherein the fenofibrate and esterase inhibitor are administered to a subject is suffering from age-related macular degeneration (AMD).

23. The method of claim 22, wherein the AMD is dry AMD.

24. The method of claim 19, wherein the fenofibrate and esterase inhibitor are administered concomitantly.

25. The method of claim 19, wherein the fenofibrate and esterase inhibitor are administered sequentially.

26. The method of any one of the preceding claims wherein fenofibrate and kaempferol are administered in a molar ratio sufficient for reducing first pass metabolism of fenofibrate in a subject having age related macular degeneration.

27. The method of any one of the preceding claims, wherein the kaempferol is from a natural source.

28. The method of claim 26, wherein the natural source is a plant or plant extract comprising kaempferol.

29. The method of claim 26, wherein the natural source or extract is green tea, capers, kale, tea, broccoli, cabbage, beans, endive, leek, tomato, strawberries or grapes.

30. The method of any one of the preceding claims that improves mitochondrial membrane potential.

31. The method of any one of the preceding claims, wherein the subject does not have diabetes.
